SEQUENCE LISTING (12 sequences)

SEQ ID NO: 1 - protein, 364 aa, Mus musculus
MPHPYPALTPEQKKELSDIAHRIVAPGKGILAADESTGSIAKRLQSIGTENTEENRRFYRQLLLTADDRVNPCIGGVILFHETLYQKADDGRPFPQVIKSKGGVVGIKVDKGVVPLAGTNGETTTQGLDGLSERCAQYKKDGADFAKWRCVLKIGEHTPSALAIMENANVLARYASICQQNGIVPIVEPEILPDGDHDLKRCQYVTEKVLAAVYKALSDHHVYLEGTLLKPNMVTPGHACTQKFSNEEIAMATVTALRRTVPPAVTGVTFLSGGQSEEEASINLNAINKCPLLKPWALTFSYGRALQASALKAWGGKKENLKAAQEEYIKRALANSLACQGKYTPSGQSGAAASESLFISNHAY

SEQ ID NO: 2 - protein, 364 aa, Homo sapiens
MPYQYPALTPEQKKELSDIAHRIVAPGKGILAADESTGSIAKRLQSIGTENTEENRRFYRQLLLTADDRVNPCIGGVILFHETLYQKADDGRPFPQVIKSKGGVVGIKVDKGVVPLAGTNGETTTQGLDGLSERCAQYKKDGADFAKWRCVLKIGEHTPSALAIMENANVLARYASICQQNGIVPIVEPEILPDGDHDLKRCQYVTEKVLAAVYKALSDHHIYLEGTLLKPNMVTPGHACTQKFSHEEIAMATVTALRRTVPPAVTGITFLSGGQSEEEASINLNAINKCPLLKPWALTFSYGRALQASALKAWGGKKENLKAAQEEYVKRALANSLACQGKYTPSGQAGAAASESLFVSNHAY

SEQ ID NO: 3 - protein, 1085 aa, Mus musculus
MANSPDAAFSSPALLRSGSVYEPLKSINLPRPDNETLWDKLDHYYRIVKSTMLMYQSPTTGLFPTKTCGGEEKSKVHESLYCAAGAWALALAYRRIDDDKGRTHELEHSAIKCMRGILYCYMRQADKVQQFKQDPRPTTCLHSVFSVHTGDELLSYEEYGHLQINAVSLFLLYLVEMISSGLQIIYNTDEVSFIQNLVFCVERVYRVPDFGVWERGSKYNNGSTELHSSSVGLAKAALEAINGFNLFGNQGCSWSVIFVDLDAHNRNRQTLCSLLPRESRSHNTDAALLPCISYPAFALDDEALFSQTLDKVIRKLKGKYGFKRFLRDGYRTPLEDPNRRYYKPAEIKLFDGIECEFPIFFLYMMIDGVFRGNLEQVKEYQDLLTPLLHQTTEGYPVVPKYYYVPADFVECEKRNPGSQKRFPSNCGRDGKLFLWGQALYIIAKLLADELISPKDIDPVQRFVPLQNQRNVSMRYSNQGPLENDLVVHVALVAESQRLQVFLNTYGIQTQTPQQVEPIQIWPQQELVKAYFHLGINEKLGLSGRPDRPIGCLGTSKIYRILGKTVVCYPIIFDLSDFYMSQDVLLLIDDIKNALQFIKQYWKMHGRPLFLVLIREDNIRGSRFNPILDMLAAFKKGIIGGVKVHVDRLQTLISGAVVEQLDFLRISDTEKLPEFKSFEELEFPKHSKVKRQSSTADAPEAQHEPGITITEWKNKSTHEILQKLNDCGCLAGQTILLGILLKREGPNFITMEGTVSDHIERVYRRAGSKKLWSVVRRAASLLNKVVDSLAPSITNVLVQGKQVTLGAFGHEEEVISNPLSPRVIKNIIYYKCNTHDEREAVIQQELVIHIGWIISNSPELFSGMLKIRIGWIIHAMEYELQVRGGDKPAVDLYQLSPSEVKQLLLDILQPQQSGRCWLNRRQIDGSLNRTPPEFYDRVWQILERTPNGIVVAGKHLPQQPTLSDMTMYEMNFSLLVEDMLGNIDQPKYRQIIVELLMVVSIVLERNPELEFQDKVDLDRLVKEAFHEFQKDESRLKEIEKQDDMTSFYNTPPLGKRGTCSYLTKVVMNSLLEGEVKPSNEDSCLVS

SEQ ID NO: 4 - protein, 1093 aa, Homo sapiens
MAGAAGLTAEVSWKVLERRARTKRSGSVYEPLKSINLPRPDNETLWDKLDHYYRIVKSTLLLYQSPTTGLFPTKTCGGDQKAKIQDSLYCAAGAWALALAYRRIDDDKGRTHELEHSAIKCMRGILYCYMRQADKVQQFKQDPRPTTCLHSVFNVHTGDELLSYEEYGHLQINAVSLYLLYLVEMISSGLQIIYNTDEVSFIQNLVFCVERVYRVPDFGVWERGSKYNNGSTELHSSSVGLAKAALEAINGFNLFGNQGCSWSVIFVDLDAHNRNRQTLCSLLPRESRSHNTDAALLPCISYPAFALDDEVLFSQTLDKVVRKLKGKYGFKRFLRDGYRTSLEDPNRCYYKPAEIKLFDGIECEFPIFFLYMMIDGVFRGNPKQVQEYQDLLTPVLHHTTEGYPVVPKYYYVPADFVEYEKNNPGSQKRFPSNCGRDGKLFLWGQALYIIAKLLADELISPKDIDPVQRYVPLKDQRNVSMRFSNQGPLENDLVVHVALIAESQRLQVFLNTYGIQTQTPQQVEPIQIWPQQELVKAYLQLGINEKLGLSGRPDRPIGCLGTSKIYRILGKTVVCYPIIFDLSDFYMSQDVFLLIDDIKNALQFIKQYWKMHGRPLFLVLIREDNIRGSRFNPILDMLAALKKGIIGGVKVHVDRLQTLISGAVVEQLDFLRISDTEELPEFKSFEELEPPKHSKVKRQSSTPSAPELGQQPDVNISEWKDKPTHEILQKLNDCSCLASQAILLGILLKREGPNFITKEGTVSDHIERVYRRAGSQKLWLAVRYGAAFTQKFSSSIAPHITTFLVHGKQVTLGAFGHEEEVISNPLSPRVIQNIIYYKCNTHDEREAVIQQELVIHIGWIISNNPELFSGMLKIRIGWIIHAMEYELQIRGGDKPALDLYQLSPSEVKQLLLDILQPQQNGRCWLNRRQIDGSLNRTPTGFYDRVWQILERTPNGIIVAGKHLPQQPTLSDMTMYEMNFSLLVEDTLGNIDQPQYRQIVVELLMVVSIVLERNPELEFQDKVDLDRLVKEAFNEFQKDQSRLKEIEKQDDMTSFYNTPPLGKRGTCSYLTKAVMNLLLEGEVKPNNDDPCLIS

SEQ ID NO: 5 - protein, 142 aa, Mus musculus
MVLSGEDKSNIKAAWGKIGGHGAEYGAEALERMFASFPTTKTYFPHFDVSHGSAQVKGHGKKVADALANAAGHLDDLPGALSALSDLHAHKLRVDPVNFKLLSHCLLVTLASHHPADFTPAVHASLDKFLASVSTVLTSKYR

SEQ ID NO: 6 - protein, 142 aa, Homo sapiens
MVLSPADKTNVKAAWGKVGAHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 7 - protein, 572 aa, Mus musculus
MSYQGKKNIPRITSDRLLIKGGKIVNDDQSFYADIYMEDGLIKQIGENLIVPGGVKTIEAHSRMVIPGGIDVHTRFQMPDQGMTSADDFFQGTKAALAGGTTMIIDHVVPEPGTSLLAAFDQWREWADSKSCCDYSLHVDITEWHKGIQEEMEALVKDHGVNSFLVYMAFKDRFQLTDSQIYEVLSVIRDIGAIAQVHAENGDIIAEEQQRILDLGITGPEGHVLSRPEEVEAEAVNRSITIANQTNCPLYVTKVMSKSAAEVIAQARKKGTVVYGEPITASLGTDGSHYWSKNWAKAAAFVTSPPLSPDPTTPDFLNSLLSCGDLQVTGSAHCTFNTAQKAVGKDNFTLIPEGTNGTEERMSVIWDKAVVTGKMDENQFVAVTSTNAAKVFNLYPRKGRISVGSDADLVIWDPDSVKTISAKTHNSALEYNIFEGMECRGSPLVVISQGKIVLEDGTLHVTEGSGRYIPRKPFPDFVYKRIKARSRLAELRGVPRGLYDGPVCEVSVTPKTVTPASSAKTSPAKQQAPPVRNLHQSGFSLSGAQIDDNIPRRTTQRIVAPPGGRANITSLG

SEQ ID NO: 8 - protein, 572 aa, Homo sapiens
MSYQGKKNIPRITSDRLLIKGGKIVNDDQSFYADIYMEDGLIKQIGENLIVPGGVKTIEAHSRMVIPGGIDVHTRFQMPDQGMTSADDFFQGTKAALAGGTTMIIDHVVPEPGTSLLAAFDQWREWADSKSCCDYSLHVDISEWHKGIQEEMEALVKDHGVNSFLVYMAFKDRFQLTDCQIYEVLSVIRDIGAIAQVHAENGDIIAEEQQRILDLGITGPEGHVLSRPEEVEAEAVNRAITIANQTNCPLYITKVMSKSSAEVIAQARKKGTVVYGEPITASLGTDGSHYWSKNWAKAAAFVTSPPLSPDPTTPDFLNSLLSCGDLQVTGSAHCTFNTAQKAVGKDNFTLIPEGTNGTEERMSVIWDKAVVTGKMDENQFVAVTSTNAAKVFNLYPRKGRIAVGSDADLVIWDPDSVKTISAKTHNSSLEYNIFEGMECRGSPLVVISQGKIVLEDGTLHVTEGSGRYIPRKPFPDFVYKRIKARSRLAELRGVPRGLYDGPVCEVSVTPKTVTPASSAKTSPAKQQAPPVRNLHQSGFSLSGAQIDDNIPRRTTQRIVAPPGGRANITSLG

SEQ ID NO: 9 - protein, 706 aa, Mus musculus
MNYLRRRLSDSNFMANLPNGYMTDLQRPQPPPPPPSAASPGATPGSATASAERASTAAPVASPAAPSPGSSGGGGFFSSLSNAVKQTTAAAAATFSEQVGGGSGGAGRGGAAARVLLVIDEPHTDWAKYFKGKKIHGEIDIKVEQAEFSDLNLVAHANGGFSVDMEVLRNGVKVVRSLKPDFVLIRQHAFSMARNGDYRSLVIGLQYAGIPSVNSLHSVYNFCDKPWVFAQMVRLHKKLGTEEFPLIDQTFYPNHKEMLSSTTYPVVVKMGHAHSGMGKVKVDNQHDFQDIASVVALTKTYATAEPFIDAKYDVRVQKIGQNYKAYMRTSVSGNWKTNTGSAMLEQIAMSDRYKLWVDTCSEIFGGLDICAVEALHGKDGRDHIIEVVGSSMPLIGDHQDEDKQLIVELVVNKMTQALPRQPQRDASPGRGSHSQSSSPGALTLGRQTSQQPAGPPAQQRPPPQGGPPQPGPGPQRQGPPLQQRPPPQGQQHLSGLGPPAGSPLPQRLPSPTAAPQQSASQATPVTQGQGRQSRPVAGGPGAPPAARPPASPSPQRQAGAPQATRQASISGPAPTKASGAPPGGQQRQGPPQKPPGPAGPTRQASQAGPGPRTGPPTTQQPRPSGPGPAGRPAKPQLAQKPSQDVPPPITAAAGGPPHPQLNKSQSLTNAFNLPEPAPPRPSLSQDEVKAETIRSLRKSFASLFSD

SEQ ID NO: 10 - protein, 705 aa, Homo sapiens
MNYLRRRLSDSNFMANLPNGYMTDLQRPQPPPPPPGAHSPGATPGPGTATAERSSGVAPAASPAAPSPGSSGGGGFFSSLSNAVKQTTAAAAATFSEQVGGGSGGAGRGGAASRVLLVIDEPHTDWAKYFKGKKIHGEIDIKVEQAEFSDLNLVAHANGGFSVDMEVLRNGVKVVRSLKPDFVLIRQHAFSMARNGDYRSLVIGLQYAGIPSVNSLHSVYNFCDKPWVFAQMVRLHKKLGTEEFPLIDQTFYPNHKEMLSSTTYPVVVKMGHAHSGMGKVKVDNQHDFQDIASVVALTKTYATAEPFIDAKYDVRVQKIGQNYKAYMRTSVSGNWKTNTGSAMLEQIAMSDRYKLWVDTCSEIFGGLDICAVEALHGKDGRDHIIEVVGSSMPLIGDHQDEDKQLIVELVVNKMAQALPRQRQRDASPGRGSHGQTPSPGALPLGRQTSQQPAGPPAQQRPPPQGGPPQPGPGPQRQGPPLQQRPPPQGQQHLSGLGPPAGSPLPQRLPSPTSAPQQPASQAAPPTQGQGRQSRPVAGGPGAPPAARPPASPSPQRQAGPPQATRQTSVSGPAPPKASGAPPGGQQRQGPPQKPPGPAGPTRQASQAGPVPRTGPPTTQQPRPSGPGPAGRPKPQLAQKPSQDVPPPATAAAGGPPHPQLNKSQSLTNAFNLPEPAPPRPSLSQDEVKAETIRSLRKSFASLFSD

SEQ ID NO: 11 - protein, 381 aa, Mus musculus
MPFSNSHNTQKLRFPAEDEFPDLSSHNNHMAKVLTPELYAELRAKCTPSGFTLDDAIQTGVDNPGHPYIMTVGAVAGDEESYDVFKDLFDPIIEERHGGYQPSDEHKTDLNPDNLQGGDDLDPNYVLSSRVRTGRSIRGFCLPPHCSRGERRAIEKLAVEALSSLDGDLSGRYYALKSMTEAEQQQLIDDHFLFDKPVSPLLLASGMARDWPDARGIWHNDNKTFLVWINEEDHLRVISMQKGGNMKEVFTRFCTGLTQIETLFKSKNYEFMWNPHLGYILTCPSNLGTGLRAGVHIKLPHLGKHEKFSEVLKRLRLQKRGTGGVDTAAVGGVFDVSNADRLGFSEVELVQMVVDGVKLLIEMEQRLEQGQAIDDLMPAQK

SEQ ID NO: 12 - protein, 381 aa, Homo sapiens
MPFSNSHNALKLRFPAEDEFPDLSAHNNHMAKVLTPELYAELRAKSTPSGFTLDDVIQTGVDNPGHPYIMTVGCVAGDEESYEVFKDLFDPIIEDRHGGYKPSDEHKTDLNPDNLQGGDDLDPNYVLSSRVRTGRSIRGFCLPPHCSRGERRAIEKLAVEALSSLDGDLAGRYYALKSMTEAEQQQLIDDHFLFDKPVSPLLLASGMARDWPDARGIWHNDNKTFLVWVNEEDHLRVISMQKGGNMKEVFTRFCTGLTQIETLFKSKDYEFMWNPHLGYILTCPSNLGTGLRAGVHIKLPNLGKHEKFSEVLKRLRLQKRGTGGVDTAAVGGVFDVSNADRLGFSEVELVQMVVDGVKLLIEMEQRLEQGQAIDDLMPAQK

(19) United States
(12) Patent Application Publication
(10) Pub. No.: US 2022/0244274 A1
(43) Pub. Date: Aug. 4, 2022

(54) QUANTITATIVE BIOMARKERS FOR ASSESSING MILD TRAUMATIC BRAIN INJURY AND METHODS OF USE THEREOF

(71) Applicants: University of Iowa Research Foundation, Iowa City, IA (US); UNITED STATES DEPARTMENT OF VETERANS AFFAIRS, Washington, DC (US)

(72) Inventors: Matthew Harper, Tipton, IA (US); Michael Anderson, Iowa City, IA (US); Kacie Meyer, Iowa City, IA (US); Laura M. Dutca, North Liberty, IA (US); John Manohar, Ames, IA (US); Randy Kardon, Iowa City, IA (US)

(21) Appl. No.: 17/591,521

(22) Filed: Feb. 2, 2022

Related U.S. Application Data
(60) Provisional application No. 63/144,926, filed on Feb. 2, 2021.

(51) Int. Cl. G01N 33/68; G01N 33/72

(52) U.S. Cl. CPC G01N 33/6896; G01N 2333/76; G01N 2800/28; G01N 33/721

(57) ABSTRACT

Disclosed here is a method of detecting traumatic brain injury in a subject, comprising collecting a biological sample from the subject; analyzing the biological sample to determine the level of at least one protein selected from ALDOA, PHKB, HBA-A1, DPYSL2, SYN1 and/or CKB; and determining whether the level of the at least one protein exceeds a predetermined threshold. In certain aspects, the method further comprises the step of administering a treatment to the subject if the at least one protein exceeds the predetermined threshold. The disclosed technology relates generally to brain injuries, and in particular to a panel of serum based biomarkers that can identify individuals with mild traumatic brain injury (TBI).

### CROSS-REFERENCE TO RELATED APPLICATION(S)

This application claims priority to U.S. Provisional Application No. 63/144,926 filed Feb. 2, 2021 and entitled “APPARATUS, SYSTEMS AND METHODS FOR QUANTITATIVE BIOMARKERS FOR ASSESSING MILD TRAUMATIC BRAIN INJURY,” which is hereby incorporated by reference in its entirety under 35 U.S.C. § 119(e).

### GOVERNMENT SUPPORT

This invention was made with government support under W81XWH-14-1-0583 awarded by the Department of Defense, and RX000952 awarded by the U.S. Department of Veterans Affairs. The government has certain rights in this invention.

### TECHNICAL FIELD

The disclosed technology relates generally to brain injuries, and in particular to a panel of serum based biomarkers that can identify individuals with mild traumatic brain injury (TBI). Discovery of serum-based biomarkers to identify individuals with TBI is important because important because no routine, easily administered diagnostic tests have been identified that can differentiate between patients with TBI. There is a current unmet need for these tests in the medical community, as TBI is often diagnosed using subjective outcomes. These needs exist in civilian accidents, soldiers and veterans that have experienced blast after combat, and athletes at the amateur, college and professional level.

### BACKGROUND

Blast-mediated traumatic brain injury (TBI) is a common condition among active and recently-active military personnel, and also affects civilian populations. Blast-mediated TBI is a traumatic event that needs both acute and chronic management, and symptoms typically manifest and progress chronically. Identification of individuals with mild TBI or TBI-induced symptoms is difficult for multiple reasons, including self-reporting of blast-exposure. In addition, improvements in protective armor have improved survivability in recent conflicts, which has resulted in an increased incidence of TBI. Even if TBI is suspected based on the reported history, a confounding factor for symptom-based diagnosis is that individuals with TBI can present with a wide constellation of symptoms which include cognitive, behavioral, neuropsychological, motor and visual impairment. Many of these symptoms may not be immediately apparent and may only manifest months to years after the initial injury, or are diagnosed post-mortem. The only existing test is based on a single protein biomarker and is unreliable. Thus, there is a significant need in the art for objective blood-based biomarkers for mild injuries that can be used to help confirm diagnosis.

### BRIEF SUMMARY

Disclosed here is a method of detecting traumatic brain injury in a subject, comprising collecting a biological sample from the subject; analyzing the biological sample to determine the level of at least one protein selected from ALDOA, PHKB, HBA-A1, DPYSL2, SYN1 and/or CKB; and determining whether the level of the at least one protein exceeds a predetermined threshold. In certain aspects, the method further comprises the step of administering a treatment to the subject if the at least one protein exceeds the predetermined threshold.

In certain implementations, the subject is determined to have a mild traumatic brain injury (mTBI) when one or more of ALDOA, PHKB, HBA-A1, DPYSL2, SYN1 and/or CKB is detectable. In further implementations, the subject is determined to have mTBI, if one or more of the biomarker proteins exceeds a level established from one or more healthy control subjects.

According to certain embodiments, the method further comprises assessing the subject via the Glasgow Coma Scale. In exemplary implementations, the method further involves performing and imaging procedure on the subject if the Glasgow Coma Score is below a predetermined threshold and one or more biomarker exceeds a predetermined threshold.

In further embodiments, the step of determining the level of at least one protein is performed by immunoassay and/or mass spectroscopy.

Further disclosed herein is a method of measuring or detecting at least one biomarker by obtaining a biological sample from a subject after an actual or suspected head injury; and measuring or detecting at least one peptide of at least one biomarker or fragment thereof selected from the group consisting of ALDOA, PHKB, HBA-A1, DPYSL2, SYN1, CKB, or any combinations thereof in the sample. In certain implementations, the subject is determined to have mTBI if amount the at least one peptide of at least one biomarker or fragment thereof measured or detected exceeds a predetermined threshold. In further implementations, the subject exceeds the predetermined threshold if the level exceeds a level established from one or more control subjects. In further implementations, the subject exceeds the predetermined threshold if the at least one peptide of at least one biomarker or fragment thereof is detectable. In certain embodiments, the step of measuring or detecting is performed by immunoassay and/or mass spectroscopy. In further embodiments, the biomarker or fragment thereof is HBA-A1.

Further disclosed herein is a method, comprising measuring or detecting a level of at least one biomarker in a biological sample obtained from a subject, wherein the at least one biomarker comprises HBA-A1, wherein measuring or detecting the level of the at least one biomarker determines whether the subject has sustained an mTBI; and administering a treatment for mTBI to the subject. In certain implementations, the subject is determined to have mTBI if HBA-A1 is detectable in the biological sample. In further implementations, the subject is determined to have mTBI if the amount of HBA-A1 exceeds the amount measured in one or more control subject by a predetermined threshold. In certain embodiments, the treatment is one or more of the group consisting of: rest, abstaining from physical activities, avoiding light, an analgesic, an anti-nausea medication, and further monitoring.

While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 shows a schematic representation of the PELS principle for generation of affinity-captured proteome/depletome used to identify TBI-biomarkers, according to certain embodiments.

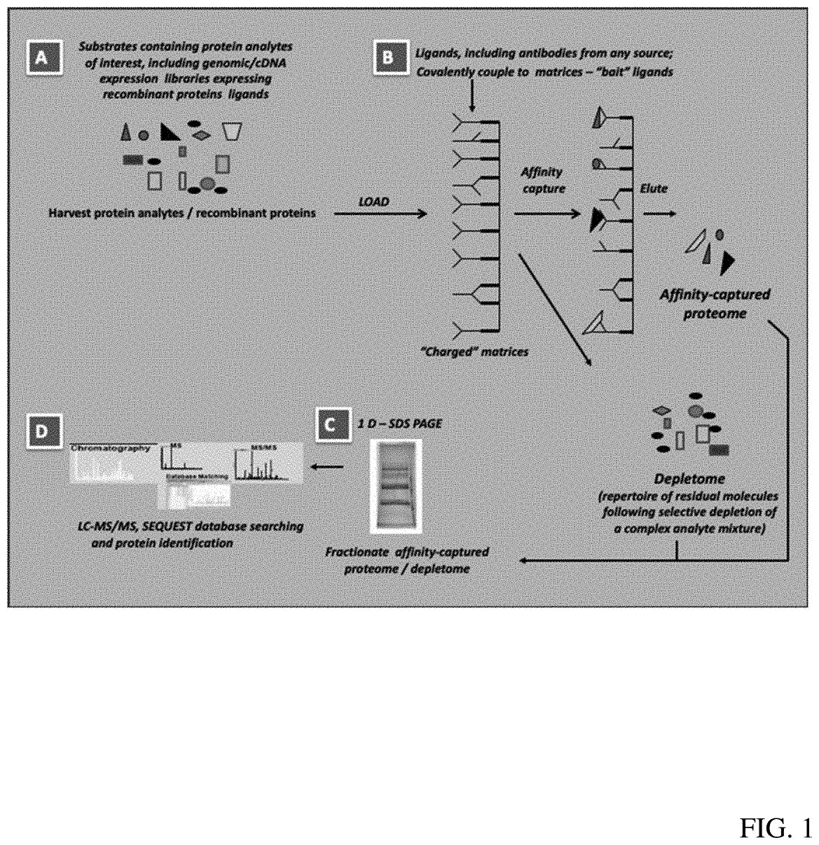

### DETAILED DESCRIPTION

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

A used herein, “subject” and “patient” as used herein interchangeably refers to any vertebrate, including, but not limited to, a mammal and a human. In some embodiments, the subject may be a human or a non-human. The subject or patient may be undergoing other forms of treatment. In some embodiments, when the subject is a human, the subject does not include any humans who have suffered a cerebrovascular accident (e.g., a stroke). In some embodiments, the subject is suspected to have sustained an injury to the head. In some embodiments, the subject is known to have sustained an injury to the head. In some embodiments, the subject is suspected to be suffering from mild, moderate or severe TBI. In some embodiments, the subject is suspected to be suffering from mild TBI.

As used herein, a “control subject” relates to a subject or subjects that have not sustained a traumatic brain injury.

As used herein, “Glasgow Coma Scale” or “GCS” as used herein refers to a 15 point scale for estimating and categorizing the outcomes of brain injury on the basis of overall social capability or dependence on others. The test measures the motor response, verbal response and eye opening response with these values:

I. Motor Response (6—Obeys commands fully; 5—Localizes to noxious stimuli; 4—Withdraws from noxious stimuli; 3—Abnormal flexion, i.e. decorticate posturing; 2—Extensor response, i.e. decerebrate posturing; and 1—No response);

II. Verbal Response (5—Alert and Oriented; 4—Confused, yet coherent, speech; 3—Inappropriate words and jumbled phrases consisting of words; 2—Incomprehensible sounds; and 1—No sounds); and

III. Eye Opening (4—Spontaneous eye opening; 3—Eyes open to speech; 2—Eyes open to pain; and 1—No eye opening).

The final score is determined by adding the values of I+II+III. The final score can be categorized into four possible levels for survival, with a lower number indicating a more severe injury and a poorer prognosis: Mild (13-15); Moderate Disability (9-12) (Loss of consciousness greater than 30 minutes; Physical or cognitive impairments which may or may resolve: and Benefit from Rehabilitation); Severe Disability (3-8) (Coma: unconscious state. No meaningful response, no voluntary activities); and Vegetative State (Less Than 3) (Sleep wake cycles; Arousal, but no interaction with environment; No localized response to pain). Moderate brain injury is defined as a brain injury resulting in a loss of consciousness from 20 minutes to 6 hours and a Glasgow Coma Scale of 9 to 12. Severe brain injury is defined as a brain injury resulting in a loss of consciousness of greater than 6 hours and a Glasgow Coma Scale of 3 to 8.

As used herein, “imaging procedure” as used herein refers to a medical test that allows the inside of a body to be seen in order to diagnose, treat, and monitor health conditions. An imaging procedure can be a non-invasive procedure that allows diagnosis of diseases and injuries without being intrusive. Examples of imaging procedures include MRI, CT scan, X-rays, positron emission tomography (PET) scan, single-photon emission computed tomography (SPECT), and diffusion tensor imaging (DTI) scan.

As used herein, “injury to the head” or “head injury” as used interchangeably herein, refers to any trauma to the scalp, skull, or brain. Such injuries may include only a minor bump on the skull or may be a serious brain injury. Such injuries include primary injuries to the brain and/or secondary injuries to the brain. Primary brain injuries occur during the initial insult and result from displacement of the physical structures of the brain. More specifically, a primary brain injury is the physical damage to parenchyma (tissue, vessels) that occurs during the traumatic event, resulting in shearing and compression of the surrounding brain tissue. Secondary brain injuries occur subsequent to the primary injury and may involve an array of cellular processes. More specifically, a secondary brain injury refers to the changes that evolve over a period of time (from hours to days) after the primary brain injury. It includes an entire cascade of cellular, chemical, tissue, or blood vessel changes in the brain that contribute to further destruction of brain tissue.

An injury to the head can be either closed or open (penetrating). A closed head injury refers to a trauma to the scalp, skull or brain where there is no penetration of the skull by a striking object. An open head injury refers a trauma to the scalp, skull or brain where there is penetration of the skull by a striking object. An injury to the head may be caused by physical shaking of a person, by blunt impact by an external mechanical or other force that results in a closed or open head trauma (e.g., vehicle accident such as with an automobile, plane, train, etc.; blow to the head such as with a baseball bat, or from a firearm), a cerebral vascular accident (e.g., stroke), one or more falls (e.g., as in sports or other activities), explosions or blasts (collectively, “blast injuries”) and by other types of blunt force trauma. In certain embodiments herein, the closed head injury does not include and specifically excludes a cerebral vascular accident, such as stroke.

As used herein, “sample”, “test sample”, “biological sample” refer to fluid sample containing or suspected of containing a mTBI biomarker. The sample may be derived from any suitable source. In some cases, the sample may comprise a liquid, fluent particulate solid, or fluid suspension of solid particles. In some cases, the sample may be processed prior to the analysis described herein. For example, the sample may be separated or purified from its source prior to analysis; however, in certain embodiments, an unprocessed sample containing a mTBI biomarker may be assayed directly. In a particular example, the source containing a mTBI biomarker is a human bodily substance (e.g., bodily fluid, blood such as whole blood, serum, plasma, urine, saliva, sweat, sputum, semen, mucus, lacrimal fluid, lymph fluid, amniotic fluid, interstitial fluid, lung lavage, cerebrospinal fluid, feces, tissue, organ, or the like).

As used herein, “treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a pharmaceutical composition to a subject that is not at the time of administration afflicted with the disease. “Preventing” also refers to preventing the recurrence of a disease or of one or more symptoms associated with such disease. “Treatment” and

The various embodiments disclosed or contemplated herein relate to six new biomarkers for the identification of subjects suffering from mTBI. Those proteins are ALDOA, PHKB, HBA-A1, DPYSL2, SYN1, and CKB (Table 1).

TABLE 1
mTBI Protein Biomarkers

| Identified | Accession | Molecular | | Gene |
|---|---|---|---|---|
| proteins | number | weight | UniProtKB | symbol |
| Fructose- | IPI00221402 | 39 kDa | P05064 | ALDOA |
| bisphosphate | | | | |
| aldolase A | | | | |
| Phosphorylase b | IPI00380735 | 124 kDa | Q7TSH2 | Phkb |
| kinase regulatory | | | | |
| subunit beta | | | | |
| Alpha globin 1 | IPI00845802 | 15 kDa | Q91VB8 | Hba-a1 |
| Dihy- | IPI00114375 | 62 kDa | O08553 | Dpysl2 |
| dropyrimidinase- | | | | |
| related protein 2 | | | | |
| Isoform Ib of | IPI00136372 | 70 kDa | O88935 | Syn1 |
| Synapsin-1 | (+1) | | | |
| Creatine kinase | IPI00136703 | 43 kDa | Q04447 | Ckb |
| B-type | | | | |

Disclosed here is a method of detecting traumatic brain injury in a subject, comprising collecting a biological sample from the subject; analyzing the biological sample to determine the level of at least one protein selected from ALDOA, PHKB, HBA-A1, DPYSL2, SYN1 and/or CKB; and determining whether the level of the at least one protein exceeds a predetermined threshold. In certain embodiments, the method involves the step determining whether at least one protein is selected from the group consisting of SEQ ID NOs: 1-12. In certain aspects, the method further comprises the step of administering a treatment to the subject if the at least one protein exceeds the predetermined threshold.

In certain implementations, treatments for mTBI include instructing the subject to rest and abstain from physical activities, especially such activities that risk further head injuries. Treatment may also involve instructing the subject to avoid light and or loud noises. Treatment may also involve administration of one or more analgesics, and/or one or more anti-nausea medication. In further embodiments, treatment for mTBI is further medical monitoring which may include but is not limited to further monitoring and/or performing an imaging procedure. Such treatments are used to assess whether mTBI may progress to a more serve TBI that may require additional intervention.

In certain implementations, the subject is determined to have mTBI when one or more of ALDOA, PHKB, HBA-A1, DPYSL2, SYN1 and/or CKB is detectable in the biological sample of the subject. In further implementations, the subject is determined to have mTBI, if one or more of the biomarker proteins exceeds a level established from one or more healthy control subjects.

According to certain embodiments, the method further comprises assessing the subject via the Glasgow Coma Scale. In exemplary implementations, the method further involves performing and imaging procedure on the subject if the Glasgow Coma Score is below a predetermined threshold and one or more biomarker exceeds a predetermined threshold.

In certain embodiments, the at least one protein is HBA-A1.

In further embodiments, the biological sample is serum.

In further embodiments, the step of determining the level of at least one protein is performed by immunoassay and/or mass spectroscopy.

Further disclosed herein is a method of measuring or detecting at least one biomarker by obtaining a biological sample from a subject after an actual or suspected head injury; and measuring or detecting at least one peptide of at least one biomarker or fragment thereof selected from the group consisting of ALDOA, PHKB, HBA-A1, DPYSL2, SYN1, CKB, or any combinations thereof in the sample. In certain implementations, the subject is determined to have mTBI if amount the at least one peptide of at least one biomarker or fragment thereof measured or detected exceeds a predetermined threshold. In further implementations, the subject exceeds the predetermined threshold if the level exceeds a level established from one or more control subjects. In further implementations, the subject exceeds the predetermined threshold if the at least one peptide of at least one biomarker or fragment thereof is detectable. In certain embodiments, the step of measuring or detecting is performed by immunoassay and/or mass spectroscopy. In further embodiments, the biomarker or fragment thereof is HBA-A1.

Further disclosed herein is a method of measuring or detecting at least one biomarker by obtaining a biological sample from a subject after an actual or suspected head injury; and measuring or detecting at least one peptide of at least one biomarker or fragment thereof selected from the group consisting of ALDOA, PHKB, HBA-A1, DPYSL2, SYN1, CKB, or any combinations thereof in the sample, wherein the at least one peptide of the at least one biomarker is selected from the group consisting of SEQ ID NOs: 1-12.

Further disclosed herein is a method, comprising measuring or detecting a level of at least one biomarker in a biological sample obtained from a subject, wherein the at least one biomarker comprises HBA-A1, wherein measuring or detecting the level of the at least one biomarker determines whether the subject has sustained an mTBI; and administering a treatment for mTBI to the subject. In certain implementations, the subject is determined to have mTBI if HBA-A1 is detectable in the biological sample. In further implementations, the subject is determined to have mTBI if the amount of HBA-A1 exceeds the amount measured in one or more control subject by a predetermined threshold. In certain embodiments, the treatment is one or more of the group consisting of: rest, abstaining from physical activities, avoiding light, an analgesic, an anti-nausea medication, and further monitoring.

In the methods described above, mTBI biomarker levels can be measured by any means, such as antibody dependent methods, such as immunoassays, protein immunoprecipitation, immunoelectrophoresis, chemical analysis, SDS-PAGE and Western blot analysis, protein immunostaining, electrophoresis analysis, a protein assay, a competitive binding assay, a functional protein assay, or chromatography or spectrometry methods, such as high-performance liquid chromatography (HPLC), mass spectrometry, or liquid chromatography-mass spectrometry (LC/MS) or capillary electrophoresis (CE)-MS, or direct infusion, or any separating front end coupled with MS. Also, the assay can be employed in clinical chemistry format such as would be known by one skilled in the art.

In some embodiments, measuring the level of a mTBI biomarker includes contacting the sample with a first specific binding element and second specific binding element. In some embodiments the first specific binding element is a capture antibody and the second specific binding element is a detection antibody. In some embodiments, measuring the level of a mTBI biomarker includes contacting the sample, either simultaneously or sequentially, in any order: (1) a capture antibody (e.g., a mTBI biomarker-capture antibody), which binds to an epitope on a mTBI biomarker or a mTBI biomarker fragment to form a capture antibody-mTBI biomarker antigen complex (e.g., mTBI biomarker-capture antibody-mTBI biomarker antigen complex), and (2) a detection antibody (e.g., TBI biomarker-detection antibody), which includes a detectable label and binds to an epitope on a TBI biomarker that is not bound by the capture antibody, to form a mTBI biomarker antigen-detection antibody complex (e.g., mTBI biomarker antigen-mTBI biomarker-detection antibody complex), such that a capture antibody-mTBI biomarker antigen-detection antibody complex (e.g., mTBI biomarker-capture antibody-mTBI biomarker antigen-mTBI biomarker-detection antibody complex) is formed, and measuring the amount or concentration of a mTBI biomarker in the sample based on the signal generated by the detectable label in the capture antibody-TBI biomarker antigen-detection antibody complex.

In some embodiments, the sample is obtained after the human subject sustained an injury to the head caused by a blast or explosion, physical shaking, blunt impact by an external mechanical or other force that results in a closed or open head trauma, one or more falls, explosions or blasts or other types of blunt force trauma.

It may be desirable to include a control. The control may be analyzed concurrently with the sample from the subject as described above. The results obtained from the subject sample can be compared to the results obtained from the control sample. Standard curves may be provided, with which assay results for the sample may be compared. Such standard curves present levels of marker as a function of assay units, i.e. fluorescent signal intensity, if a fluorescent label is used. Using samples taken from multiple donors, standard curves can be provided for reference levels of a TBI biomarker in normal healthy tissue, as well as for “at-risk” levels of the mTBI biomarker in tissue taken from donors, who may have one or more of the characteristics set forth above.

Provided herein is a kit, which may be used for assaying or assessing a test sample for one or more mTBI biomarkers and/or fragments thereof. The kit comprises at least one component for assaying the test sample for a mTBI biomarker and instructions for assaying the test sample for a TBI biomarker. For example, the kit can comprise instructions for assaying the test sample for a mTBI biomarker by immunoassay (e.g., chemiluminescent microparticle immunoassay) or by mass spectrometry assay (e.g., PRM-MS or MRM/SRM-MS). Instructions included in kits can be affixed to packaging material or can be included as a package insert. While the instructions are typically written or printed materials they are not limited to such. Any medium capable of storing such instructions and communicating them to an end user is contemplated by this disclosure.

The at least one component may include at least one composition comprising one or more isolated antibodies or antibody fragments thereof that specifically bind to a mTBI biomarker. The antibody may be a mTBI biomarker detection antibody and/or capture antibody.

Alternatively or additionally, the kit can comprise a calibrator or control (e.g., purified, and optionally lyophilized, mTBI biomarker) and/or at least one container (e.g., tube, microtiter plates or strips, which can be already coated with an anti-mTBI biomarker antibody) for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e. reagents, standards, buffers, diluents, etc., which are necessary to perform the assay. The instructions also can include instructions for generating a standard curve.

The kit may further comprise reference standards for quantifying a mTBI biomarker. The reference standards may be employed to establish standard curves for interpolation and/or extrapolation of mTBI biomarker concentrations. Standards cans include proteins or peptide fragments composed of amino acids residues or N15 stable isotopic labeled proteins or peptide fragments for various analytes, as well as standards for sample processing, including standards involving spikes in proteins and quantitative peptides. In some embodiments, the reference standards for a mTBI biomarker can correspond to the 99th percentile derived from a healthy reference population. Such reference standards can be determined using routine techniques known in the art.

Any antibodies, which are provided in the kit, such as recombinant antibodies specific for a mTBI biomarker, can incorporate a detectable label, such as a fluorophore, radioactive moiety, enzyme, biotin/avidin label, chromophore, chemiluminescent label, or the like, or the kit can include reagents for labeling the antibodies or reagents for detecting the antibodies (e.g., detection antibodies) and/or for labeling the analytes (e.g., mTBI biomarker) or reagents for detecting the analyte (e.g., mTBI biomarker). The antibodies, standard peptides or peptide fragments, calibrators, and/or controls can be provided in separate containers or pre-dispensed into an appropriate assay format, for example, into microtiter plates,

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays,

The kit can also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. The kit can further include containers for holding or storing a sample (e.g., a container or cartridge for a urine, whole blood, plasma, or serum sample). Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like.

If the detectable label is at least one acridinium compound, the kit can comprise at least one acridinium-9-carboxamide, at least one acridinium-9-carboxylate aryl ester, or any combination thereof. If the detectable label is at least one acridinium compound, the kit also can comprise a source of hydrogen peroxide, such as a buffer, solution, and/or at least one basic solution. If desired, the kit can contain a solid phase, such as a magnetic particle, bead, test tube, microtiter plate, cuvette, membrane, scaffolding molecule, film, filter paper, disc, or chip.

If desired, the kit can further comprise one or more components, alone or in further combination with instructions, for assaying the test sample for another analyte, which can be a biomarker, such as a biomarker of traumatic brain injury or disorder.

### Examples

The following examples are put forth so as to provide those of ordinary skill in the art with a complete disclosure and description of certain examples of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

The purpose of this research was to seek candidates for serum-based biomarkers of TBI, and to identify protein changes after TBI. To identify thalamic proteins differentially or uniquely associated with blast exposure, we utilized an antibody-based affinity-capture strategy (referred to as “proteomics-based analysis of depletomes”; PAD) to deplete thalamic lysates from blast-treated mice of endogenous thalamic proteins found in control mice. Analysis of this “depletome” detected 75 proteins with unique identifications.

To identify blast-associated proteins eliciting production of circulating autoantibodies, serum antibodies of blast-treated mice were immobilized, and their immunogens subsequently identified by proteomic analysis of proteins specifically captured by them following incubation with thalamic lysates (a variant of a strategy referred to as “proteomics-based expression library screening”; PELS). This analysis identified 46 blast-associated immunogenic proteins, including 6 shared in common with the PAD analysis (ALDOA, PHKB, HBA-A1, DPYSL2, SYN1, and CKB) which are appropriate for biomarker development.

### Methods Used to Identify Biomarkers for Blast-Mediated TBI

### I. Proteomics-Based Expression Library Screening (PELS).

The overall strategy followed a published PELS protocol, with variations to identify host thalamus proteins shed in body fluids following blast-mediated injury. First, “bait” polyclonal antibodies (bait PAbs) were generated from the pooled sera of TBI-mice (8 weeks post blast) and were covalently coupled to TiTrap NHS-activated columns (1 ml; GE Healthcare Life Sciences) creating “charged columns”. Next, pooled thalamic protein extracts from TBI-mice (4 weeks post blast) containing the analytes of interest were subjected to immunoaffinity capture by passage through the charged columns. The captured proteins were then eluted and subjected to tandem mass spectrometry for identification. Elutions of the same extracts loaded on NHS columns charged with bait PAbs affinity purified from sera collected from untreated mice and on NHS columns without covalently coupled polyclonal antibodies, but quenched active groups (“uncharged”) served as controls for assessing both specificity of bait PAbs and nonspecific adsorption to the column matrix.

### II. Proteomics-based Analysis of Depletomes (PAD).

The term “depletome” refers to the complement of interesting molecules resident in a complex mixture, following selective depletion of irrelevant components. To derive the depletome of the thalamus from blast-exposed mice, bait polyclonal antibodies were generated in chickens (IgY) against proteins from pooled thalami of sham-mice (C57BL/6J Male mice, 8 weeks of age at the beginning of the study) using the services of a commercial vendor (Ayes Labs, OR), and affinity purified using anti-chicken IgY polyclonal generated in goats.

The bait IgY-polyclonal antibodies (titer assessed to be >1:10,000 in dot immunoblotting against 2 μg of the immunogen mixture) were then covalently coupled to Dynabeads M-280 Tosylactivated (Invitrogen/Life Technologies, CA) and HiTrap NHS-activated columns (1 ml; GE Healthcare Life Sciences) per manufacturer guidelines. The thalamus protein extracts from TBI-mice (complex mixture; 5 mg total protein in 5 mls of PBS [pH 7.4]) were reacted first with charged Dynabeads M-280 Tosylactivated and then passed through charged HiTrap NHS-activated columns per manufacturer guidelines.

This process of selective depletion of confounding proteins from the complex mixture and the simultaneous enrichment for relevant proteins resulted in a depletome constituted by proteins that were either differentially (i.e., produced in larger amounts in thalami of TBI-mice than in those of untreated mice, defined as an increase of 1 or more identified peptides compared to untreated mice) or uniquely expressed in thalami of TBI-mice 4 weeks post injury. The proteins comprising the depletome were processed and subjected to tandem mass spectrometry for identification.

TABLE 2
Novel Biomarkers for Blast-mediated TBI

| | | | Number | | Number | | |
|---|---|---|---|---|---|---|---|
| | | | of unique | | of unique | | |
| | | | peptides in | Number | peptides | | |
| | | | thalamus of | of unique | identified as | | |
| | Accession | Molecular | untreated | peptides in | immunogenic | | Gene |
| Identified proteins | number | weight | mouse | depletome | with PELS | UniProtKB | symbol |
| Fructose- | IPI00221402 | 39 kDa | 0 | 2 | 11 | P05064 | ALDOA |
| bisphosphate | | | | | | | |
| aldolase A | | | | | | | |
| Phosphorylase b | IPI00380735 | 124 kDa | 0 | 1 | 1 | Q7TSH2 | Phkb |
| kinase regulatory | | | | | | | |
| subunit beta | | | | | | | |
| Alpha globin 1 | IPI00845802 | 15 kDa | 1 | 2 | 7 | Q91VB8 | Hba-a1 |
| Dihydropyrimidinase- | IPI00114375 | 62 kDa | 2 | 6 | 1 | O08553 | Dpysl2 |
| related protein 2 | | | | | | | |
| Isoform Ib of | IPI00136372 | 70 kDa | 3 | 6 | 2 | O88935 | Syn1 |
| Synapsin-1 | (+1) | | | | | | |
| Creatine kinase B- | IPI00136703 | 43 kDa | 4 | 8 | 1 | Q04447 | Ckb |
| type | | | | | | | |

Although the disclosure has been described with reference to preferred embodiments, persons skilled in the art will recognize that changes may be made in form and detail without departing from the spirit and scope of the disclosed apparatus, systems and methods.

SEQUENCE LISTING

SEQ ID No. 1
ALDOA
MPHPYPALTPEQKKELSDIAHRIVAPGKGILAADESTGSIAKRLQSIGTENTEENRRFYR
QLLLTADDRVNPCIGGVILFHETLYQKADDGRPFPQVIKSKGGVVGIKVDKGVVPLAGTN
GETTTQGLDGLSERCAQYKKDGADFAKWRCVLKIGEHTPSALAIMENANVLARYASICQQ
NGIVPIVEPEILPDGDHDLKRCQYVTEKVLAAVYKALSDHHVYLEGTLLKPNMVTPGHAC
TQKFSNEEIAMATVTALRRTVPPAVTGVTFLSGGQSEEEASINLNAINKCPLLKPWALTF
SYGRALQASALKAWGGKKENLKAAQEEYIKRALANSLACQGKYTPSGQSGAAASESLFIS
NHAY
SEQ ID No. 2
ALDOA (HS)
MPYQYPALTPEQKKELSDIAHRIVAPGKGILAADESTGSIAKRLQSIGTENTEENRRFYR
QLLLTADDRVNPCIGGVILFHETLYQKADDGRPFPQVIKSKGGVVGIKVDKGVVPLAGTN
GETTTQGLDGLSERCAQYKKDGADFAKWRCVLKIGEHTPSALAIMENANVLARYASICQQ
NGIVPIVEPEILPDGDHDLKRCQYVTEKVLAAVYKALSDHHIYLEGTLLKPNMVTPGHAC
TQKFSHEEIAMATVTALRRTVPPAVTGITFLSGGQSEEEASINLNAINKCPLLKPWALTF
SYGRALQASALKAWGGKKENLKAAQEEYVKRALANSLACQGKYTPSGQAGAAASESLFVS
NHAY
SEQ ID No. 3
PHKB
MANSPDAAFSSPALLRSGSVYEPLKSINLPRPDNETLWDKLDHYYRIVKSTMLMYQSPTT
GLFPTKTCGGEEKSKVHESLYCAAGAWALALAYRRIDDDKGRTHELEHSAIKCMRGILYC
YMRQADKVQQFKQDPRPTTCLHSVFSVHTGDELLSYEEYGHLQINAVSLFLLYLVEMISS
GLQIIYNTDEVSFIQNLVFCVERVYRVPDFGVWERGSKYNNGSTELHSSSVGLAKAALEA
INGFNLFGNQGCSWSVIFVDLDAHNRNRQTLCSLLPRESRSHNTDAALLPCISYPAFALD
DEALFSQTLDKVIRKLKGKYGFKRFLRDGYRTPLEDPNRRYYKPAEIKLFDGIECEFPIF
FLYMMIDGVFRGNLEQVKEYQDLLTPLLHQTTEGYPVVPKYYYVPADFVECEKRNPGSQK
RFPSNCGRDGKLFLWGQALYIIAKLLADELISPKDIDPVQRFVPLQNQRNVSMRYSNQGP
LENDLVVHVALVAESQRLQVFLNTYGIQTQTPQQVEPIQIWPQQELVKAYFHLGINEKLG
LSGRPDRPIGCLGTSKIYRILGKTVVCYPIIFDLSDFYMSQDVLLLIDDIKNALQFIKQY
WKMHGRPLFLVLIREDNIRGSRFNPILDMLAAFKKGIIGGVKVHVDRLQTLISGAVVEQL
DFLRISDTEKLPEFKSFEELEFPKHSKVKRQSSTADAPEAQHEPGITITEWKNKSTHEIL
QKLNDCGCLAGQTILLGILLKREGPNFITMEGTVSDHIERVYRRAGSKKLWSVVRRAASL
LNKVVDSLAPSITNVLVQGKQVTLGAFGHEEEVISNPLSPRVIKNIIYYKCNTHDEREAV
IQQELVIHIGWIISNSPELFSGMLKIRIGWIIHAMEYELQVRGGDKPAVDLYQLSPSEVK
QLLLDILQPQQSGRCWLNRRQIDGSLNRTPPEFYDRVWQILERTPNGIVVAGKHLPQQPT
LSDMTMYEMNFSLLVEDMLGNIDQPKYRQIIVELLMVVSIVLERNPELEFQDKVDLDRLV
KEAFHEFQKDESRLKEIEKQDDMTSFYNTPPLGKRGTCSYLTKVVMNSLLEGEVKPSNED
SCLVS
SEQ ID NO. 4
PHKB (HS)
MAGAAGLTAEVSWKVLERRARTKRSGSVYEPLKSINLPRPDNETLWDKLDHYYRIVKSTL
LLYQSPTTGLFPTKTCGGDQKAKIQDSLYCAAGAWALALAYRRIDDDKGRTHELEHSAIK
CMRGILYCYMRQADKVQQFKQDPRPTTCLHSVFNVHTGDELLSYEEYGHLQINAVSLYLL
YLVEMISSGLQIIYNTDEVSFIQNLVFCVERVYRVPDFGVWERGSKYNNGSTELHSSSVG
LAKAALEAINGFNLFGNQGCSWSVIFVDLDAHNRNRQTLCSLLPRESRSHNTDAALLPCI
SYPAFALDDEVLFSQTLDKVVRKLKGKYGFKRFLRDGYRTSLEDPNRCYYKPAEIKLFDG
IECEFPIFFLYMMIDGVFRGNPKQVQEYQDLLTPVLHHTTEGYPVVPKYYYVPADFVEYE
KNNPGSQKRFPSNCGRDGKLFLWGQALYIIAKLLADELISPKDIDPVQRYVPLKDQRNVS
MRFSNQGPLENDLVVHVALIAESQRLQVFLNTYGIQTQTPQQVEPIQIWPQQELVKAYLQ
LGINEKLGLSGRPDRPIGCLGTSKIYRILGKTVVCYPIIFDLSDFYMSQDVFLLIDDIKN
ALQFIKQYWKMHGRPLFLVLIREDNIRGSRFNPILDMLAALKKGIIGGVKVHVDRLQTLI
SGAVVEQLDFLRISDTEELPEFKSFEELEPPKHSKVKRQSSTPSAPELGQQPDVNISEWK
DKPTHEILQKLNDCSCLASQAILLGILLKREGPNFITKEGTVSDHIERVYRRAGSQKLWL
AVRYGAAFTQKFSSSIAPHITTFLVHGKQVTLGAFGHEEEVISNPLSPRVIQNIIYYKCN
THDEREAVIQQELVIHIGWIISNNPELFSGMLKIRIGWIIHAMEYELQIRGGDKPALDLY
QLSPSEVKQLLLDILQPQQNGRCWLNRRQIDGSLNRTPTGFYDRVWQILERTPNGIIVAG
KHLPQQPTLSDMTMYEMNFSLLVEDTLGNIDQPQYRQIVVELLMVVSIVLERNPELEFQD
KVDLDRLVKEAFNEFQKDQSRLKEIEKQDDMTSFYNTPPLGKRGTCSYLTKAVMNLLLEG
EVKPNNDDPCLIS
SEQ ID No. 5
HBA-A1
MVLSGEDKSNIKAAWGKIGGHGAEYGAEALERMFASFPTTKTYFPHFDVSHGSAQVKGHG
KKVADALANAAGHLDDLPGALSALSDLHAHKLRVDPVNFKLLSHCLLVTLASHHPADFTP
AVHASLDKFLASVSTVLTSKYR
SEQ ID No. 6
HBA-A1 (HS)
MVLSPADKTNVKAAWGKVGAHAGEYGAEALERMFLSFPTTKTYFPHFDLSHGSAQVKGHG
KKVADALTNAVAHVDDMPNALSALSDLHAHKLRVDPVNFKLLSHCLLVTLAAHLPAEFTP
AVHASLDKFLASVSTVLTSKYR
SEQ ID No. 7
DPYSL2
MSYQGKKNIPRITSDRLLIKGGKIVNDDQSFYADIYMEDGLIKQIGENLIVPGGVKTIEA
HSRMVIPGGIDVHTRFQMPDQGMTSADDFFQGTKAALAGGTTMIIDHVVPEPGTSLLAAF
DQWREWADSKSCCDYSLHVDITEWHKGIQEEMEALVKDHGVNSFLVYMAFKDRFQLTDSQ
IYEVLSVIRDIGAIAQVHAENGDIIAEEQQRILDLGITGPEGHVLSRPEEVEAEAVNRSI
TIANQTNCPLYVTKVMSKSAAEVIAQARKKGTVVYGEPITASLGTDGSHYWSKNWAKAAA
FVTSPPLSPDPTTPDFLNSLLSCGDLQVTGSAHCTFNTAQKAVGKDNFTLIPEGTNGTEE
RMSVIWDKAVVTGKMDENQFVAVTSTNAAKVFNLYPRKGRISVGSDADLVIWDPDSVKTI
SAKTHNSALEYNIFEGMECRGSPLVVISQGKIVLEDGTLHVTEGSGRYIPRKPFPDFVYK
RIKARSRLAELRGVPRGLYDGPVCEVSVTPKTVTPASSAKTSPAKQQAPPVRNLHQSGFS
LSGAQIDDNIPRRTTQRIVAPPGGRANITSLG
SEQ ID No. 8
DPYSL2 (HS)
MSYQGKKNIPRITSDRLLIKGGKIVNDDQSFYADIYMEDGLIKQIGENLIVPGGVKTIEA
HSRMVIPGGIDVHTRFQMPDQGMTSADDFFQGTKAALAGGTTMIIDHVVPEPGTSLLAAF
DQWREWADSKSCCDYSLHVDISEWHKGIQEEMEALVKDHGVNSFLVYMAFKDRFQLTDCQ
IYEVLSVIRDIGAIAQVHAENGDIIAEEQQRILDLGITGPEGHVLSRPEEVEAEAVNRAI
TIANQTNCPLYITKVMSKSSAEVIAQARKKGTVVYGEPITASLGTDGSHYWSKNWAKAAA
FVTSPPLSPDPTTPDFLNSLLSCGDLQVTGSAHCTFNTAQKAVGKDNFTLIPEGTNGTEE
RMSVIWDKAVVTGKMDENQFVAVTSTNAAKVFNLYPRKGRIAVGSDADLVIWDPDSVKTI
SAKTHNSSLEYNIFEGMECRGSPLVVISQGKIVLEDGTLHVTEGSGRYIPRKPFPDFVYK
RIKARSRLAELRGVPRGLYDGPVCEVSVTPKTVTPASSAKTSPAKQQAPPVRNLHQSGFS
LSGAQIDDNIPRRTTQRIVAPPGGRANITSLG
SEQ ID No. 9
SYN1
MNYLRRRLSDSNFMANLPNGYMTDLQRPQPPPPPPSAASPGATPGSATASAERASTAAPV
ASPAAPSPGSSGGGGFFSSLSNAVKQTTAAAAATFSEQVGGGSGGAGRGGAAARVLLVID
EPHTDWAKYFKGKKIHGEIDIKVEQAEFSDLNLVAHANGGFSVDMEVLRNGVKVVRSLKP
DFVLIRQHAFSMARNGDYRSLVIGLQYAGIPSVNSLHSVYNFCDKPWVFAQMVRLHKKLG
TEEFPLIDQTFYPNHKEMLSSTTYPVVVKMGHAHSGMGKVKVDNQHDFQDIASVVALTKT
YATAEPFIDAKYDVRVQKIGQNYKAYMRTSVSGNWKTNTGSAMLEQIAMSDRYKLWVDTC
SEIFGGLDICAVEALHGKDGRDHIIEVVGSSMPLIGDHQDEDKQLIVELVVNKMTQALPR
QPQRDASPGRGSHSQSSSPGALTLGRQTSQQPAGPPAQQRPPPQGGPPQPGPGPQRQGPP
LQQRPPPQGQQHLSGLGPPAGSPLPQRLPSPTAAPQQSASQATPVTQGQGRQSRPVAGGP
GAPPAARPPASPSPQRQAGAPQATRQASISGPAPTKASGAPPGGQQRQGPPQKPPGPAGP
TRQASQAGPGPRTGPPTTQQPRPSGPGPAGRPAKPQLAQKPSQDVPPPITAAAGGPPHPQ
LNKSQSLTNAFNLPEPAPPRPSLSQDEVKAETIRSLRKSFASLFSD
SEQ ID No. 10
SYN1 (HS)
MNYLRRRLSDSNFMANLPNGYMTDLQRPQPPPPPPGAHSPGATPGPGTATAERSSGVAPA
ASPAAPSPGSSGGGGFFSSLSNAVKQTTAAAAATFSEQVGGGSGGAGRGGAASRVLLVID
EPHTDWAKYFKGKKIHGEIDIKVEQAEFSDLNLVAHANGGFSVDMEVLRNGVKVVRSLKP
DFVLIRQHAFSMARNGDYRSLVIGLQYAGIPSVNSLHSVYNFCDKPWVFAQMVRLHKKLG
TEEFPLIDQTFYPNHKEMLSSTTYPVVVKMGHAHSGMGKVKVDNQHDFQDIASVVALTKT
YATAEPFIDAKYDVRVQKIGQNYKAYMRTSVSGNWKTNTGSAMLEQIAMSDRYKLWVDTC
SEIFGGLDICAVEALHGKDGRDHIIEVVGSSMPLIGDHQDEDKQLIVELVVNKMAQALPR
QRQRDASPGRGSHGQTPSPGALPLGRQTSQQPAGPPAQQRPPPQGGPPQPGPGPQRQGPP
LQQRPPPQGQQHLSGLGPPAGSPLPQRLPSPTSAPQQPASQAAPPTQGQGRQSRPVAGGP
GAPPAARPPASPSPQRQAGPPQATRQTSVSGPAPPKASGAPPGGQQRQGPPQKPPGPAGP
TRQASQAGPVPRTGPPTTQQPRPSGPGPAGRPKPQLAQKPSQDVPPPATAAAGGPPHPQL
NKSQSLTNAFNLPEPAPPRPSLSQDEVKAETIRSLRKSFASLFSD
SEQ ID No. 11
CKB
MPFSNSHNTQKLRFPAEDEFPDLSSHNNHMAKVLTPELYAELRAKCTPSGFTLDDAIQTG
VDNPGHPYIMTVGAVAGDEESYDVFKDLFDPIIEERHGGYQPSDEHKTDLNPDNLQGGDD
LDPNYVLSSRVRTGRSIRGFCLPPHCSRGERRAIEKLAVEALSSLDGDLSGRYYALKSMT
EAEQQQLIDDHFLFDKPVSPLLLASGMARDWPDARGIWHNDNKTFLVWINEEDHLRVISM
QKGGNMKEVFTRFCTGLTQIETLFKSKNYEFMWNPHLGYILTCPSNLGTGLRAGVHIKLP
HLGKHEKFSEVLKRLRLQKRGTGGVDTAAVGGVFDVSNADRLGFSEVELVQMVVDGVKLL
IEMEQRLEQGQAIDDLMPAQK
SEQ ID No. 12
CKB (HS)
MPFSNSHNALKLRFPAEDEFPDLSAHNNHMAKVLTPELYAELRAKSTPSGFTLDDVIQTG
VDNPGHPYIMTVGCVAGDEESYEVFKDLFDPIIEDRHGGYKPSDEHKTDLNPDNLQGGDD
LDPNYVLSSRVRTGRSIRGFCLPPHCSRGERRAIEKLAVEALSSLDGDLAGRYYALKSMT
EAEQQQLIDDHFLFDKPVSPLLLASGMARDWPDARGIWHNDNKTFLVWVNEEDHLRVISM
QKGGNMKEVFTRFCTGLTQIETLFKSKDYEFMWNPHLGYILTCPSNLGTGLRAGVHIKLP
NLGKHEKFSEVLKRLRLQKRGTGGVDTAAVGGVFDVSNADRLGFSEVELVQMVVDGVKLL
IEMEQRLEQGQAIDDLMPAQK

What is claimed is:

1. A method of detecting mild traumatic brain injury (mTBI) in a subject, comprising:
a. collecting a biological sample from the subject;
b. analyzing the biological sample to determine the level of at least one protein selected from ALDOA, PHKB, HBA-A1, DPYSL2, SYN1 and/or CKB; and
c. determining whether the level of the at least one protein exceeds a predetermined threshold.

2. The method of claim 1, further comprising the step of administering a treatment to the subject if the at least one protein exceeds the predetermined threshold.

3. The method of claim 2, wherein the subject exceeds the predetermined threshold if the level of the at least one protein is detectable.

4. The method of claim 2, wherein the subject exceeds the predetermined threshold if the level of the at least one protein exceeds a level established from one or more control subjects.

5. The method of claim 2, further comprising assessing the subject via the Glasgow Coma Scale.

6. The method of claim 2, wherein the treatment is one or more of the group consisting of: rest, abstaining from physical activities, avoiding light, medication for relief of a headache or migraine, anti-nausea medication, and further monitoring.

7. The method of claim 5, further comprising performing and imaging procedure on the subject if the Glasgow Coma Score is below a predetermined threshold.

8. The method of claim 1, wherein the at least one protein is HBA-A1.

9. The method of claim 1, wherein the biological sample is serum.

10. The method of claim 1, wherein the step of determining the level of at least one protein is performed by immunoassay and/or mass spectroscopy.

11. A method of measuring or detecting at least one biomarker, the method comprising:
a. obtaining a biological sample from a subject after an actual or suspected head injury; and
b. measuring or detecting at least one peptide of at least one biomarker or fragment thereof selected from the group consisting of ALDOA, PHKB, HBA-A1, DPYSL2, SYN1, CKB, or any combinations thereof in the sample.

12. The method of claim 11, wherein the subject is determined to have mTBI if amount the at least one peptide of at least one biomarker or fragment thereof measured or detected exceeds a predetermined threshold.

13. The method of claim 12, wherein the subject exceeds the predetermined threshold if the level exceeds a level established from one or more control subjects.

14. The method of claim 11, the subject exceeds the predetermined threshold if the at least one peptide of at least one biomarker or fragment thereof is detectable.

15. The method of claim 11, wherein the step of measuring or detecting is performed by immunoassay and/or mass spectroscopy.

16. The method of claim 11, wherein the biomarker or fragment thereof is HBA-A1.

17. A method, comprising:
a. measuring or detecting a level of at least one biomarker in a biological sample obtained from a subject, wherein the at least one biomarker comprises HBA-A1, wherein measuring or detecting the level of the at least one biomarker determines whether the subject has sustained an mTBI; and
b. administering a treatment for mTBI to the subject.

18. The method of claim 17, wherein the subject is determined to have mTBI if HBA-A1 is detectable in the biological sample.

19. The method of claim 17, wherein the subject is determined to have mTBI if the amount of HBA-A1 exceeds the amount measured in one or more control subject by a predetermined threshold.

20. The method of claim 17, wherein the treatment is one or more of the group consisting of: rest, abstaining from physical activities, avoiding light, an analgesic, an anti-nausea medication, and further monitoring.
